(19) United States
(12) Patent Application Publication
(10) Pub. No.: US 2006/0216823 A1
(43) Pub. Date: Sep. 28, 2006

(54) Method for obtaining an enriched population of siRNA-expressing cells

(75) Inventors: Vicente Planelles, Salt Lake City, UT (US); Erik S. Zimmerman, Salt Lake City, UT (US); Jason L. DeHart, Salt Lake City, UT (US)

Correspondence Address: TRASK BRITT, P.O. BOX 2550, SALT LAKE CITY, UT, 84110, US

(73) Assignee: University of Utah Research Foundation, Salt Lake City, UT (US)

(21) Appl. No.: 11/445,682

(22) Filed: Jun. 1, 2006

Related U.S. Application Data
(63) Continuation of application No. PCT/US04/41714, filed on Dec. 10, 2004.
(60) Provisional application No. 60/528,567, filed on Dec. 10, 2003.

(51) Int. Cl. C40B 40/02; C40B 40/08; C07H 21/02; C12N 15/09

(52) U.S. Cl. 435/455; 536/23.1

(57) ABSTRACT

Problems with transience of siRNA-mediated knock-down and transfection efficiency have limited the scope of RNAi-based experiments. The invention provides a tool for employing RNAi more efficiently and effectively by integrating RNAi expression with methods of cell enrichment.

### CROSS-REFERENCE TO RELATED APPLICATIONS

This application is a continuation of PCT International Patent Application No. PCT/US2004/041714, filed on Dec. 10, 2004, designating the United States of America, and published, in English, as PCT International Publication No. WO 2005/059102 A3 on Jun. 30, 2005, which application claims priority to U.S. Provisional Patent Application Ser. No. 60/528,567, filed on Dec. 10, 2003, the contents of the entirety of each of which are incorporated herein by this reference. This application also claims the benefit of the previously identified U.S. Provisional Patent Application No. 60/528,567 filed Dec. 10, 2003.

### STATEMENT REGARDING FEDERALLY SPONSORED RESEARCH OR DEVELOPMENT

This research of the present invention was supported in part by the National Institutes of Health (AI054188). The US government may have rights in this invention.

### TECHNICAL FIELD

The invention relates to biotechnology generally and, more particularly, to enrichment of double-stranded RNA expression.

### BACKGROUND

The ability to specifically down-regulate gene expression is a powerful means of gene function analysis in many model systems. Until recently, the only available method of targeted gene down-regulation in mammalian systems has been expensive and cumbersome gene knockout technology in mice. However, the characterization of RNA interference (RNAi) in C. elegans and plants led to the discovery of a similar system in mammalian cells. RNAi is the phenomenon by which double-stranded, small interfering RNAs (siRNAs) target the cognate mRNA for degradation by the RNA Induced Silencing Complex (RISC), thus suppressing gene expression at the translational level (G. J. Hannon (2002), RNA interference, Nature 418(6894):244-51).

However, there are several limitations to effectively applying RNAi technology in mammalian systems. First, mammalian cells lack the RNA-dependent RNA polymerase, present in C. elegans and plants, which facilitates amplification and sustained expression of the interfering RNA signal (P. J. Paddison and G. J. Hannon (2002), RNA interference: the new somatic cell genetics?, Cancer Cell 2(1):17-23 (hereinafter Paddison 2002); and Hannon 2002). Because of this, the effect of transfected siRNAs in mammalian cells is transient, restricting the temporal window available for gene function analysis. The development of polIII promoter-based expression vectors that express short hairpin RNAs (shRNAs) resembling double-stranded siRNAs may be used to overcome siRNA transience (Brummelkamp et al. (2002), A system for stable expression of short interfering RNAs in mammalian cells, Science 296(5567):550-3). Such vectors allow for a sustained RNAi effect by transient or stable transfection of the vector producing the RNAi effect. However, this approach is limited by transfection efficiency variability in different cell culture conditions or cell types.

In addition to transfection, a library of siRNA molecules may be introduced into the appropriate host cells using a viral approach. For example, lentiviral vectors may be used. However, the construction and preparation of a library of viral vectors requires a major investment in time and money. In addition, it is also possible that the viral vector itself will modulate a cellular response that may be undesirable. Finally, viral based vectors, e.g., retrovirus-based vectors, cannot be used for the stable expression of siRNAs in all cell types, this would require the added time and expense of moving siRNA-encoding inserts to different vectors.

The present invention overcomes these limitations and provides compositions and methods that, for example, may save significant amounts of both time and money.

### SUMMARY OF THE INVENTION

The invention provides a technique to circumvent the limitations of transfection efficiency while retaining desirable sustained RNAi expression. The invention relates to a method of obtaining cells capable of expressing inhibitory RNA by introducing at least one nucleic acid into a cell, wherein the at least one nucleic acid is capable of expressing an RNAi molecule and a separation marker; expressing the separation marker; sorting the cell based on expression of the separation marker; and expressing the RNAi molecule in an amount sufficient to inhibit expression of the target gene.

The invention also relates to targeting cellular, exogenous, viral and transgenes for RNA inhibition. In one embodiment, the invention relates to transfection of mammalian cells while retaining desirable sustained RNAi expression.

The invention further relates to sorting cells based on expression of a separation marker. Exemplary embodiments include sorting by fluorescence activated cell sorting and magnetic cell separation.

The invention also relates to a method of enriching a population of mammalian cells having an RNAi sequence by providing eukaryotic, e.g., mammalian, cells containing a target gene, into which a construct capable of expressing an RNA and a separation marker are introduced, wherein the RNA contains a double-stranded region of the molecule with a first region having a sequence which corresponds to a nucleotide sequence of the target gene and a second region having a sequence which is complementary to the first region, wherein the first and the second regions of the RNA hybridize to each other to form a double-stranded RNA molecule; expressing the separation marker, wherein expression of the separation marker is indicative of the presence of the double-stranded RNA molecule; and sorting the eukaryotic cells expressing the separation marker, thereby enriching for the eukaryotic cells having the double-stranded RNA sequence.

The invention also relates to one or more recombinant nucleic acids having a separation marker and a promoter operably linked to a nucleotide sequence having a sequence which is complementary to a target gene product. In an exemplary embodiment, the nucleotide sequence comprises a first region which is complementary to the target gene product and a second region which is complementary to the first region.

In an exemplary embodiment, the recombinant nucleic acid is a vector or expression vector. One exemplary embodiment of an expression vector is pHYPER.

In another exemplary embodiment, the invention relates to a library of siRNA sequences, wherein the library may be random sequences or a related set of sequences. In another exemplary embodiment, the invention relates to a method of preparing and/or enriching cells transfected with a library of related siRNA sequences or a random set of siRNA sequences. In another exemplary embodiment, the invention provides the ability to enrich the population transfected with a library of siRNA sequences.

In another exemplary embodiment, the invention relates to a recombinant nucleic acid, comprising a nucleic acid having a first region including one or more unknown nucleotide positions, a second region capable of forming a loop and a third region comprising the complement of the one or more unknown nucleotide positions, wherein the nucleic acid is capable of forming a stem-loop structure.

The invention further relates to a recombinant nucleic acid comprising at least one nucleic acid having a means for producing a separation marker and a means for producing a ribonucleotide sequence which is complementary to a target gene product.

### BRIEF DESCRIPTION OF THE DRAWINGS

FIG. 1 is a map of the pMACS-K/k-H1-Rad17 plasmid showing the shRNA sequence (SEQ ID NO:1 being the top strand and SEQ ID NO:2 being the complement to the bottom strand).

FIGS. 2A, B, and D illustrate that transfection efficiencies of 49% and 42% were achieved for pMACS-K/k-H1 and pMACS-K/k-H1-Rad17, respectively compared to HeLa control as indicated by α-K/k.II-FITC binding. FIGS. 2C and E illustrate that cell populations were enriched to purities of 97% and 98% for pMACS-K/k-H1 and pMACS-K/k-H1-Rad17, respectively.

FIG. 3 shows that Rad17 protein levels are significantly reduced in pMACS-K/k-H1-Rad17 expressing cells relative to those expressing no RNAi (pMACS-K/k-H1) at least 72 hours post-transfection.

FIG. 4 illustrates a method of constructing a library of RNAi sequences in a plasmid of the invention utilizing Cre/Lox recombination to transfer a library of sequences from one source to another.

### DETAILED DESCRIPTION OF THE INVENTION

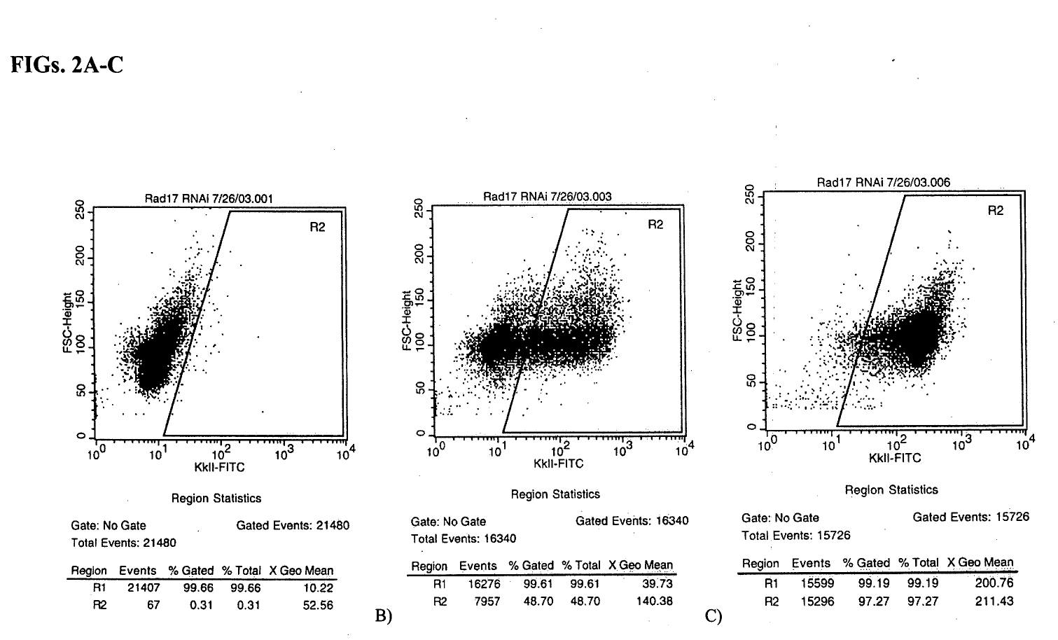

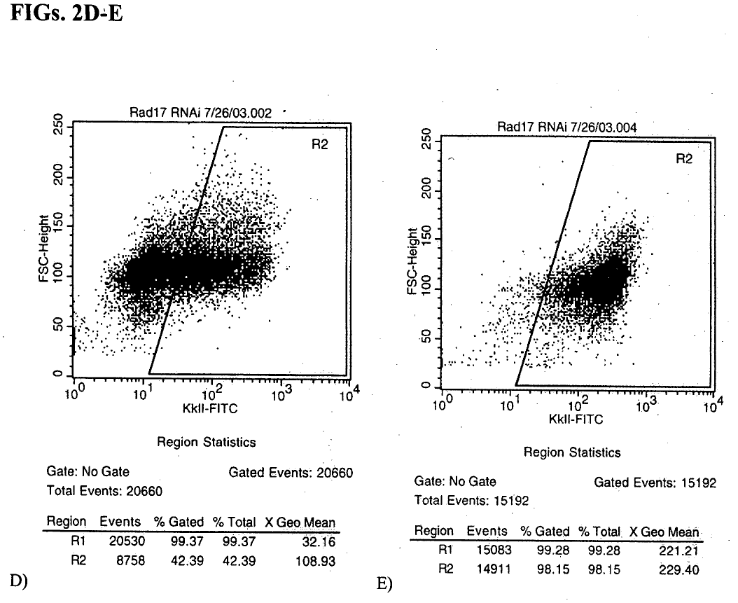

Problems with the transience of siRNA-mediated knock-down and transfection efficiency, for example, transfection of polIII-based RNAi expression vectors, have limited the scope of RNAi-based experiments, for example, in mammalian-based model systems. The invention provides a tool for employing RNAi more efficiently and effectively by integrating RNAi expression with methods of cell enrichment. In an exemplary embodiment, the invention provides a method of isolating a population of cells, for example, at least 97% pure, for expression of the pMACS-K/k-H1-Rad17 plasmid (FIG. 2). Furthermore, expression of shRNA from this plasmid can suppress expression of a mammalian gene product, and this inhibition can be maintained for at least 72 hours after transfection of the pMACS-K/k-H1-Rad17 plasmid (FIG. 3). Thus, the invention demonstrates the successful application of cell separation, for example, pMACS-K/k-H1, as a means of enriching a population for the presence of RNAi.

Two groups, P. J. Paddison et al. (2004), A Resource for Large-scale RNA-interference-based Screens in Mammals, Nature 428:427-431, and K. Berns et al. (2004), A Large-scale RNAi Screen in Human Cells Identifies New Components of the p53 Pathway, Nature 428:431-437 (see also Andrew Fraser (2004), RNA Interference: Human Genes Hit the Big Screen, Nature 428:375-378), have utilized a retroviral vector to conduct large-scale screening, e.g., genome-wide. However, both groups have utilized retroviral vectors and such vectors have a number of disadvantages that are overcome by the present invention.

shRNAs often inhibit or knock-down target genes to differing extents, hence multiple, e.g., greater than two, three, or four shRNAs may be generated for each gene. Such multiple coverage may provide an internal control, and/or allow comparison of knock-downs having differing strengths, e.g., strong and weak, allowing analysis comparable to classical genetic approaches.

RNA interference may be produced by an number of different RNA inhibitors, as used herein “RNA inhibitor,” “inhibiting RNA” and similar phrases mean RNA sequences capable of producing an effect referred to as RNA interference, which is believed to result from the sequence-specific cleavage of mRNAs, preventing translation of functional gene products, inhibiting RNAs include, shRNA, siRNA, dsRNA and other RNAi products.

The invention relates to a population of cells that may be enriched for production of an siRNA. A person of skill in the art may, for example, test or analyze several target sequences in order to optimize the knock-down effect or compare the effects. Additionally, a person of skill in the art may modulate knock-down efficacy due to such factors as target mRNA abundance, expression level, turnover rate and/or by protein half-life (Elbashir et al. (2001), Duplexes of 21-nucleotide RNAs mediate RNA interference in cultured mammalian cells, Nature 411(6836):494-8).

The pMACS-K/k-H1 system of RNA interference can be applied as a means or method of studying suppression of gene function, for example, in mammalian cells. This technique may be useful for RNA interference in cell lines that are difficult to transfect. With a very minimal level of transfection, one can isolate a highly enriched population of shRNA-expressing cells. Additionally, the method facilitates experiments requiring an extended period of gene suppression.

The pMACS-K/k-H1 system of RNAi delivery is an improvement on the current technology that may expand the ability to perform RNAi-based experiments. Further, cells separated based on expression of shRNA or other RNAi, may be useful for treatment of disease, for example, by ex vivo gene therapy. The methods of the invention may be used with any cell, including, but not limited to, eukaryotic cells such as mammalian cells or plant cells.

In an exemplary embodiment, the invention provides a technique to circumvent the limitations of transfection efficiency while retaining the desirable sustained RNAi expression of a polIII vector-based system. By cloning the polIII H1 promoter into a pMACS-K/k.II plasmid (Miltenyi Biotech., Cologne, Germany) for transfected cell separation, cell populations 98% positive for shRNA expression were reproducibly isolated. Using this system, significant knock-down of a mammalian gene expression was demonstrated, up to 72 hours after RNAi-expressing plasmid transfection. Thus, this system facilitates more consistent, sustained and enhanced RNA interference. Furthermore, it is now possible to perform effective RNAi experiments in cell lines that have previously been difficult to transfect with either synthetic siRNA, shRNA or double-stranded expression vectors.

The invention provides a method wherein the RNAi inhibition signal is increased and/or the noise is decreased for a population of cells. For example, transfection of primary cells or lymphocytes may result in a transfection efficiency of less than about 5%. Thus, without selection the signal obtained from the RNAi knockout is likely to be overwhelmed by the noise from the non-transfected cells. Using the methods and compositions of the invention, a population of cells may be transformed or transfected, selected to enrich for the transformed or transfected cells and the selected cells assayed. Thereby removing the background from the non-transfected (non-transformed) cells. Thus, the invention allows RNAi studies in cells having a low transfection or transformation efficiency. For example, a transformation or transfection efficiency of less than 50%, less than 40%, less than 30%, less than 20% or less than 10% may benefit from the invention. For example, HeLa cells may have a transfection efficiency of approximately 30%. Using the method of the invention the noise due to non-transfected cells may be reduced, thereby improving a downstream assay, such as, a phenotypic and/or genotypic analysis.

The reduction in noise due to non-transfected cells is particularly advantageous when screening and/or analyzing a large number of constructs, such as a library of siRNA constructs.

Sorting or enriching for transfected cells may be performed by fluorescence activated cell sorting (FACS) (Gross et al. (1995), Model Study Detecting Breast Cancer Cells in Peripheral Blood Mononuclear Cells at Frequencies as low as 10⁻⁷, Proc. Natl. Acad. Sci. USA), immuno-affinity cell separation techniques (Lebkowski et al. (1992), Rapid isolation of CD34+ cells from PB of autologous transplant patient, Blood 80 (suppl.) 1:527; and U.S. Pat. No. 5,912,177), affinity-based separation methods (for example, a streptavidin affinity column for a biotin label, magnetic microparticles coupled to antibodies (Shpall et al. (1991), Bone Marrow Transplantation 7:145) and High gradient magnetic cell sorting (Miltenyi et al. (1990), High Gradient Magnetic Cell Sorting with MACS, Cytometry 11:231-239), dielectrophoretic/gravitational field-flow fractionation and centrifugation. An overview of cell separation techniques is provided by Cell Separation Methods and Applications, Recktenwald et al., eds. (1998). As will be recognized by a person of ordinary skill in the art, cell sorting or enrichment may utilize separation markers that are recognized by antibodies, photometric systems or the like, such as fluorescent molecules and chemiluminescent markers.

In an exemplary embodiment, separation antibodies are coupled to a magnetic reagent, such as a paramagnetic micro- or nano-particle (microparticle or nanoparticle). Magnetic cell separation is a means/method whereby target cells may be labeled with a magnetic marker and then selectively retained or excluded through exposure to a magnetic field. Examples of such methods can be found in Kantor et al. (1998, Magnetic Cell Sorting with Colloidal Superparamagnetic Particles, in Cell Separation Methods and Applications); Gee (1998, Immunomagnetic Cell Separation Using Antibodies and Superparamagnetic Microspheres in Cell Separation Methods and Applications); and U.S. Pat. No. 5,411,863. For high gradient magnetic separation, typically a heterogeneous suspension, containing selected cells bound to magnetic markers, is passed through a column, allowing cells having a magnetic marker to adhere magnetically to a column or a paramagnetic matrix within the column. The remainder of the suspension is eluted, leaving the desired, magnetized cells bound to the column. When the magnetic field is removed, the bound cells can be eluted. U.S. Pat. No. 4,452,773 describes the preparation of magnetic iron-dextran microparticles and provides a summary describing the various means of preparing particles suitable for attachment to biological materials. A description of polymeric coatings for magnetic particles may be found in DE 3720844 and U.S. Pat. No. 5,385,707. Methods to prepare superparamagnetic particles are described in U.S. Pat. No. 4,770,183. Magnetic cell separation includes, but is not limited to, diamagnetic, paramagnetic, ferromagnetic and superparamagnetic materials (see, U.S. Pat. No. 5,385,707). Thus, the invention includes magnetic separation using a range of magnetic properties. The extent of magnetization which is acquired by a particle is a function of magnetic moment, volume of the particle and the applied magnetic field. For example, the higher the magnetic moment and the smaller the volume, the higher the magnetization.

In another exemplary embodiment, separation markers or antibodies are, or are coupled to, a fluorochrome. The transfected or transformed population of cells may then be sorted by fluorescence activated cell sorting. In yet another exemplary embodiment, the transfected or transformed cells are sorted by a method commonly referred to as panning. For example, antibodies are coupled to a solid support and the transfected or transformed population is exposed to the antibodies on the solid support. Subsequently, unbound cells are removed by washing while the cells expressing the separation marker, which may be bound directly or indirectly by the attached antibodies, will remain bound to the solid support. The bound cells may then be eluted at the desired time.

Separation markers may be found on the cell surface (for example, the plasma membrane or cell wall), extracellular matrix, in the cytoplasm, cellular organelles, cellular organelle compartments or organelle membranes, such as the nuclear membrane, or extracellularly (see, United States Patent Application 20020182645). However, the separation marker preferably should be accessible to the sorting means or method without disruption of the cell. For example, green fluorescence protein (see U.S. Pat. No. 6,319,669) or Beta galactosidase (e.g., using fluorescein digalactoside (FDG)) may be expressed from a vector, incorporated into the nuclear membrane, and the cells sorted by fluorescence-activated cell sorting (FACS) or flow cytometry (see Anderson et al. (1996), Proc. Natl. Acad. Sci. USA 93:8508-8511). In an exemplary embodiment, the separation marker is any molecule not expressed, or expressed at suitably low levels, in the cell population to be sorted, for example, one or more intercellular adhesion molecules (ICANs) or fragments thereof may be used. In yet another embodiment, a recombinant chimeric molecule may be used. For example, a fragment of an IgG immunoglobulin (e.g., a mouse heavy chain or fragment thereof) may be fused to a cell surface protein. In another embodiment, the separation molecule may alter the size, shape or other properties of a cell. For example, the separation marker may alter the forward and/or side scatter properties of the cell. Thus, the separation markers of the invention include any means or molecule capable of identifying a cell carrying the RNAi construct.

The separation marker may act directly or indirectly and may be detected directly or indirectly. A person of ordinary skill in the art, using the guidance provided herein, will recognize that the particular separation marker or separation method used is not crucial to the invention and may be any appropriate method and/or molecule, or fragment of a molecule, sufficient to identify the cell and allow separation from cells lacking the separation marker. A separation marker may be combined with a selectable marker (for example, antibiotic resistance markers such as puromycin-resistance and ampicillin resistance). Separation markers differ from selection markers in that a separation marker does not require the application of lethal drugs to the cells. Further, separation markers offer significantly more choices than does the use of selection markers. For example, multiple separation markers may be used and cells may be sorted into those having all markers or subsets of the markers. Thus, significantly more flexibility is offered with separation markers. In addition, the presence of one or more selection markers has raised concerns that cells containing these markers (typically antibiotic resistance genes) could transfer the resistance to bacteria or other organisms, making antibiotic medicines less effective. In contrast, a separation marker may be used without such concern.

The exact method for detecting a separation marker is not critical to the practice of the invention, and a number of alternatives are known in the art. For example, separation antibodies may be directly coupled to detectable particles. Indirect coupling can be accomplished by several methods. The antibodies may be coupled to one member of a high affinity binding system (e.g., biotin) and the particles attached to the other member (e.g., avidin). One may also use second stage antibodies that recognize species-specific epitopes of the antibodies, e.g. anti-mouse Ig, anti-rat Ig, etc. Indirect coupling methods allow the use of a single magnetically coupled entity, e.g. antibody, avidin, magnetoliposome, etc., with a variety of separation antibodies.

In one embodiment, hapten-specific second stage antibodies coupled to detectable particles may be used (e.g., magnetic particles or fluorochromes). As used herein “detectable particles” include magnetic reagents and fluorescent molecules. The hapten-specific antibodies may have an affinity of at least about 100 μM for the hapten. The antibodies may be conjugated to an appropriate hapten. Suitable haptens include digoxin, digoxigenin, FITC, dinitrophenyl, nitrophenyl, etc. Methods for conjugation of a hapten or detectable particle to an antibody are known in the art.

Immuno-magnetic selection has been used to select cells expressing a marker, for example, a surface marker, transiently transfected cells containing a plasmid(s) encoding a selectable marker, and after uptake and expression of the plasmid(s), the transfected cells are immuno-adsorbed to antibody coated magnetic beads. In a single round of magnetic selection, it is possible to enrich the cell population more than seven-fold in the case of co-transfection. See Aileen Constans (2000), Field of Dreams: Innovations in Magnetic Particle Technology Advance Many Fields, The Scientist 14(13):23; and Partington (1999), A novel method of cell separation based on dual parameter immunomagnetic cell selection, J. Immunol. Methods. 223(2): 195-205.

Antibodies of the invention may include enzyme conjugated antibodies (e.g., horseradish peroxidase, phosphatase, etc.), haptenated antibodies (e.g., biotin conjugates, digoxigenin conjugates, etc.) or a fluorochrome conjugated antibody (e.g., phycoerythrin, FITC, rhodamine, Texas red, allophycocyanin, etc.). The antibodies may have specificity for any antigen useful in the separation of a subpopulation. Reagents may also include blocking agents that reduce non-specific labeling (e.g., Fc receptor blocking reagent, peroxidase blocking reagent, etc.). For example, a cocktail of digoxigenin-coupled antibodies may be used in combination with anti-digoxigenin antibody coupled to magnetic particles, which may be followed by labeling with a fluorochrome conjugated antibody directed to the anti-hapten antibody.

A library of siRNA sequences, includes, but is not limited to, a library of random sequences or a library of related set of sequences. A related set of sequences may comprise siRNA sequences capable of inhibiting one or more members of a gene family or subfamily, examples of gene families and the methods to categorize genes into a family or subfamily are well known in the art. As will be recognized by a person of ordinary skill in the art, gene families or subfamilies are common and the list of such families is continually expanding. As an illustrative example, the 5-hydroxytryptamine (5-HT) receptor family is known to have at least seven subfamilies (5-HT₁₋₇). Using the present invention, it is possible to construct a library of RNAi sequences, transfect a population of cells with the library, enrich the population for those transfected with an RNAi sequence, and to screen the enriched population. The ability to enrich the transfected population provides advantages when conducting screens using a library of random sequences (e.g., genome-wide screening) or a related set of sequences (e.g., gene family screening and/or analysis, gene subfamily screening and/or analysis, screening and/or analyzing a protein class. As will be recognized by a person of ordinary skill in the art, the ability to enrich the population may provide significant advantages where a large number of constructs are to be introduced and screened and/or analyzed.

Transfection of siRNA is a critical step in gene silencing experiments. A difficulty encountered with all transfection techniques is the transfection efficiency. For example, if a pool of cells is transfected at less than 100% efficiency, which is almost always the case, any assay will become difficult to interpret or require significantly more time and/or reagents, all of which increase the cost. As will be recognized by a person of ordinary skill in the art, this aspect of transfection drives the need for appropriate transfection efficiency controls. In another exemplary embodiment, the invention relates to a method of preparing and/or enriching cells transfected with a library of related siRNA sequences or a random set of siRNA sequences. The library of siRNA sequences may be produced by methods known in the art, for example, those described in Hutvagner et al., A Cellular Function for the RNA-interference Enzyme Dicer in the Maturation of the Let-7 Small Temporal RNA, Science 293(5531):834-838; International Patent Publication WO 99/64582; International Patent Publication WO 00/49035; International Patent Publication WO 96/29097; Pasquinelli et al. (2000), Conservation of the Sequence and Temporal Expression of Let-7 Heterochronic Regulatory RNA, Nature 408(6808);86-89; Yu Jenn-Yah et al. (2002), RNA Interference by Expression of Short-Interfering RNAs and hairpin RNAs in Mammalian Cells, Proc. Natl. Acad. Sci. USA 99(9):6047-6052; Raykov et al. (2002), Transient Suppression of Transgene Expression by Means of Antisense Oligonucleotides: A Method for the Production of Toxin-transducing Recombinant Viruses, Gene Therapy 9(5):358-362; International Patent Publication WO 03/020931 A2 to Gert-Jan Arts et al.; and Gert-Jan Arts et al. (2003), Adenoviral Vectors Expressing siRNAs for Discovery and Validation of Gene Function, Genome Research 13(10):2325-2332, which are incorporated by reference.

Screening and/or analysis of a library of siRNA molecules is typically done using a viral approach. However, such an approach is disadvantageous in that it requires a major investment in time to generate the virus stocks. This approach imposes additional costs in both time and direct costs (e.g., equipment and materials). In addition, care must be exercised that the use of virus does not modulate a cellular response that is undesirable. Hence, care must be used in selecting the appropriate viral vector, and it may not be possible to effectively use a viral vector in some cases.

A cell population may be cultured in media appropriate for the particular cells. To improve recovery, sorting by methods such as FACS may be performed using blank Hank's media, or another appropriate media, as the sheath fluid. Samples to be sorted by methods such as FACS may be held in a rich medium that is most suitable to them. High serum concentrations may be advisable due to dilution in the collection vial by sheath fluid. The collection vials are typically maintained at the optimal temperature and contain media for preservation of cell viability. In addition, processing of the sorted cells as soon as possible after completion of the sorting may help maintain cell viability.

In another embodiment, the cells of the invention may be sorted multiple times. In particular, when the transformation efficiency is low, for example, less than about 10%, or the desired purity of the sorted culture is high, for example, greater than 90%, the cells may be sorted two or more times. In another embodiment, the cells are separated using two or more separation markers in one or more sorting steps.

Dead cells may be excluded from the sorted population by methods known in the art, for example, by addition (labeling) of an appropriate amount of propidium iodide (PI) or 7-aminoactinomycin D.

The inhibitory RNA molecule of the invention may be of any length appropriate and desirable for a particular purpose. For example, siRNA is typically about 18 to about 26 nucleotides in length, with length typically measured by the length of the sequence complementary to the target sequence (e.g., the mRNA) (for example, see McCaffrey et al. (2003), Inhibition of Hepatitis B Virus in Mice by RNA Interference, Nat. Biotechnol. 21(6):639-644). Double-stranded RNA may be from about 18 to thousands of nucleotides in length. Double-stranded RNA is typically cleaved either in vitro or in vivo by an RNase, such as the RNase III referred to as “dicer,” to produce siRNAs (for example, see U.S. Pat. No. 6,506,559). However, longer double-stranded RNA molecules may trigger a dsRNA-activated protein kinase (PKR) response in a cell. PKR phosphorylates EIF-2α, which induces a generalized inhibition of translation. In addition, dsRNA may activate the 2′5′ oligoadenylate polymerase/RNase L system to repress IκB, leading to cell death via apoptosis. A person of ordinary skill in the art may use methods known in the art to prevent or circumvent such a cellular response (e.g., using cells lacking a PKR response).

An shRNA may include a loop sequence of between about four to about 26 nucleotides. The loop sequence need only be sufficiently long to allow the molecule to fold back on itself to produce the double-stranded “stem” structure (an intramolecular hybridization).

The double-stranded RNAi may comprise one or more strands of polymerized ribonucleotide. The double-stranded structure may be formed by one or more self-complementary or complementary RNA strands.

RNA containing a nucleotide sequences identical to a portion of the target gene is typically preferred for inhibition. However, the invention has the advantage of being able to tolerate sequence variations that might be expected due to genetic mutation, strain polymorphism, or evolutionary divergence. Therefore, insertions, deletions, and mutations, relative to the target sequence, are also effective for inhibition. The duplex region of the RNA may be defined functionally as a nucleotide sequence that is capable of hybridizing with a portion of the target gene transcript. Thus, a nucleotide sequence from a portion of the target gene may be chosen to produce inhibitory RNA. RNAi is effective in producing inhibition of gene expression and may be used to inhibit many different types of target genes (U.S. Pat. No. 6,506,559). As described herein, the RNAi sequence need not be 100% identical to the target gene to produce inhibition (Jackson et al. (2003), Expression profiling reveals off-target gene regulation by RNAi, Nat. Biotechnol. 21(6):635-637). For example, sequence differences at the 3′ end of the siRNA sense strand (5′ end of the antisense strand) relative to the target gene may still permit effective RNA inhibition.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art, for example, Asubel, 1992; Wetmur and Davidson, 1968.

Thus, as herein used, the term “stringent conditions” means hybridization will occur only if there is at least 80%, preferably at least 90%, more preferable 95% and most preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA, followed by washing the filters in 0.1×SSC at about 65° C.

Methods of attaching a nucleic acid to a membrane or support are well known in the art. For example, methods and representative membranes and supports may be found in Ausubel et al., CURRENT PROTOCOLS IN MOLECULAR BIOLOGY (John Wiley & Sons, Inc., 2004). In addition, screening assays, methods of library preparation and other such methods and associated material are known in the art and may be found in Ausubel et al. Methods and materials relating to immunology, including antibodies and their use, may be found in Bierer et al., CURRENT PROTOCOLS IN IMMUNOLOGY (John Wiley & Sons, Inc., 2004), and Harlow and Lane, ANTIBODIES: A LABORATORY MANUAL (Cold Spring Harbor Laboratory Press, 1988); methods and materials relating to cytometry may be found in Robinson et al., CURRENT PROTOCOLS IN CYTOMETRY (John Wiley & Sons, Inc., 2004).

As used herein, the term “target gene” means a gene or nucleic acid sequence derived from a cell or a virus (such as, an endogenous gene or sequence), an exogenous gene or sequence (such as, a gene, allele, or sequence not typically present in the genome), a transgene or transgenic sequence (such as, a gene construct or sequence introduced into the cell, either integrated or extrachromosomally), or a gene or a sequence from a pathogen which is capable of infecting an organism from which the cell is derived. A target gene may include more than one nucleic acid sequence, for example, a target gene may include one or more members of a gene family or subfamily, for example, a target gene may be one or more members of a previously unidentified gene family. Likewise, a target gene may include nucleic acid sequences, such as pseudogenes, isoforms, and/or splice variants.

Inhibition of gene expression refers to the observable decrease in the level of protein and/or RNA product (gene product) from a target gene. Targeted inhibition refers to the ability to inhibit the target gene without manifest effects on other genes of the cell. Inhibition can be measured by methods well known in the art. For example, RNAi inhibition in a cell line or whole organism may be assayed by use of a reporter or other assayable marker (e.g., a drug resistance gene). Such reporter genes or assays are well known in the art.

Depending on the assay, quantification of the amount of gene expression allows one to determine a degree of inhibition. Quantification of gene expression in a cell may show inhibition of target mRNA or translation of target protein. For example, a gene product mRNA may be detected with a hybridization probe, or translated polypeptide may be detected with an antibody raised against the polypeptide sequence.

In an exemplary embodiment, the nucleic acid of the invention is a vector. The vectors of the invention may be one or more nucleic acids (for example, nucleic acids capable of homologous recombination or Cre/Lox mediated recombination). The sequence of interest may be transiently, conditionally or constitutively expressed. Further, chromosomally integrated vectors can produce a stably transformed or transfected cell line. Vectors for forming such stable cell lines include, without limitation, those described in U.S. Pat. No. 6,025,192 and PCT publication WO/9812339.

Promoters may be incorporated into a nucleic acid or vector of the invention as a means to initiate transcription. Suitable promoters include any nucleotide sequence capable of initiating transcription under appropriate conditions. Suitable promoters include, without limitation, polIII promoters; polII promoters (see Paddison et al. (2002), Stable suppression of gene expression by RNAi in mammalian cells, Proc. Natl. Acad. Sci. U.S.A. 99:1443), such as the Gal4 promoter, let858, SERCA, UL6, myo-2 or myo-3, Gal4p binding sites and/or Pho5; polI promoters; viral promoters, such as T7, T3, and SP6, adenoviral promoters, the cytomegalovirus immediate early promoter, and the major operator and/or promoter regions of phage λ; yeast mating factor promoters (a or α); those disclosed in U.S. Pat. No. 6,537,786, the polyhedron or p10 promoter of the baculovirus system and other sequences known to control the expression of genes and any combination thereof. A person of ordinary skill in the art may use any known or discovered promoter in combination with the invention. Promoters may be, for example, minimal, inducible, constitutive, tissue-specific, rheostatic, stress-responsive, or combinations thereof.

Inducible promoters are promoters that initiate increased levels of transcription from DNA to which they are operably linked in response to some change in cellular conditions, for example, the presence or absence of a nutrient or a change in temperature. Inducible promoters (tet, hormone receptors, and so on) may be used to, for example, facilitate gene-silencing analyses by allowing the temporary suppression of normally lethal knockouts (e.g., “essential genes”) and aid in dissecting the sequential or temporal constraints of certain cellular phenomena. Furthermore, inducible vectors may be used, for example, to induce expression of the sequence of interest at a desirable time. For example, the sequence of interest may be under the control of a promoter derived from a gene up-regulated in response to infection (e.g., Myb-type transcription factor, a late embryogenesis-abundant protein, a root-specific gene (i.e., TobRB7), D-ribulose 5-phosphate 3-epimerase, or a 20S proteasome α-subunit) by a pest or virus, thereby inducing expression of the dsRNA in response to infection. Inducible promoters are known by persons of ordinary skill in the art.

The cell having the target gene may be derived from any organism. Such organisms include a plant, animal, protozoan, bacterium, virus, or fungus. The plant may be a monocot, dicot or gymnosperm; the animal may be a vertebrate or invertebrate. Fungi include organisms in both the mold and yeast morphologies. In an exemplary embodiment, the invention relates to the use of the method for the study of mammalian cells, such as primary cells and lymphocytes.

There are several methods available for cell transfection and transformation. For example, cells may be transfected by methods including, but not limited to, calcium phosphate, DEAE-dextran, cationic polymers, cationic dendrimer, viral methods, particle bombardment, hydrodynamic and cationic lipids. The method of transfection or transformation is not critical to the invention and may be appropriately chosen by a person of ordinary skill in the art. For examples of transfection and transformation methods and materials see Bonifacino et al., CURRENT PROTOCOLS IN CYTOMETRY (John Wiley & Sons, Inc., 2004).

### EXAMPLE I

A plasmid, pHYPER, was constructed by inserting the polIII H1 promoter into a 5′ EcoRI site and 3′ BglII site of pMACS-K/k.II (Miltenyi Biotech.).

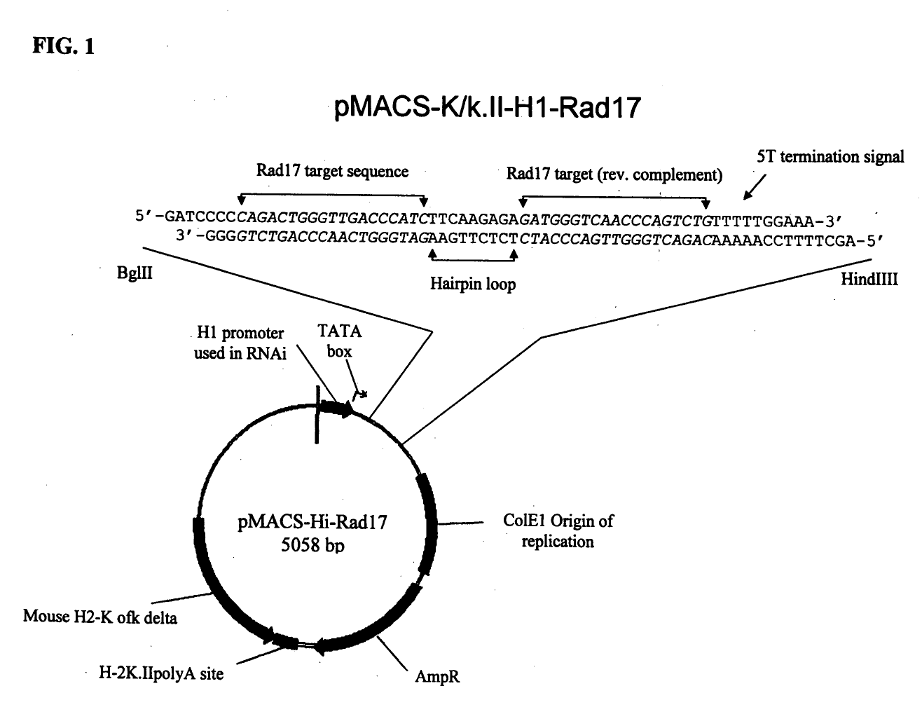

A shRNA sequence targeted to the 5′UTR of the Rad17 mRNA (Zou et al. (2002), Regulation of ATR substrate selection by Rad17-dependent loading of Rad9 complexes onto chromatin, Genes Dev. 16(2):198-208) was designed. This RNAi target sequence was incorporated into the hairpin sequence to be expressed from the H1 promoter (FIG. 1). The H1 promoter was cloned into pMACS-K/k.II so that shRNAs could be expressed in transfected cells. Next, an oligonucleotide duplex encoding for an RNA hairpin sequence targeting the 5′ untranslated region of the Rad17 mRNA (Zou, Cortez et al. 2002) was inserted downstream of the H1 promoter (FIG. 1), using 5′ BglII and 3′ HindIII sites.

Cell Culture and Transfection: HeLa cells were cultured in DMEM supplemented with 10% FBS and 1% PSG at 37° C., 5% CO2. Plasmid DNA was transfected using 10 μg DNA/10⁷ cells at ∞Ω, 975 mF, 310 V. Cells were re-cultured in fresh media for 24 hours post-transfection.

Magnetic Sorting and Flow Cytometry: Cells were detached using 2 mM EDTA in PBS 24 hours after transfection. These cells were then incubated with α-K/k.II conjugated magnetic beads at a concentration of 10 μl beads/10⁷ cells for 15 minutes and washed with MACS buffer (1×PBS, 0.5% BSA, 2 mM EDTA). Approximately 10⁵ cells were taken and incubated with α-K/k.II-FITC antibody at a concentration of 1:200 in FACS buffer (1× PBS, 2% fetal bovine serum, 0.5 mM EDTA) for ten minutes, washed with FACS buffer, fixed with 2% paraformaldehyde in PBS and analyzed for transfection efficiency by one-color flow cytometry. The remainder of the magnetic bead-bound cells were sorted using the double-positive selection program on an autoMACS magnetic cell sorter (Miltenyi Biotech.). The cells positive for expression of the separation marker were put back into culture and allowed to recover for 24 hours. Approximately 10⁵ of the positive cells were incubated with α-K/k.II-FITC antibody as described above for flow cytometric analysis of sorting efficiency.

Hela cells were transfected with pMACS-K/k-H1-Rad17 and pMACS-K/k-H1 by electroporation. Twenty-four hours after transfection, the cells were detached, incubated with α-K/k.II magnetic beads and sorted by autoMACS. Fractions of the cell populations before and after sorting were incubated with α-K/k.II-FITC antibody for analysis of transfection and autoMACS sorting efficiencies by flow cytometry. One-color flow cytometry showed that initial transfection efficiencies of 49% and 42% for pMACS-K/k-H1 and pMACS-K/k-H1-Rad17 were achieved, respectively, when compared to a mock transfected control (FIG. 1). Analysis of the positively sorted cell population showed the high purity of our positively selected cell population. The post-sort population of pMACS-K/k-H1 transfected cells was 97% pure for K/k.II expression (FIG. 2). 98% of the positively sorted population of pMACS-K/k-H1-Rad17 transfected cells expressed K/k.II and presumably shRNA directed at Rad17 mRNA (FIG. 2).

Western Blotting: Cells were harvested 24 and 48 hours after autoMACS sorting and resuspended in Laemmli's Buffer at a concentration of 10⁶ cells/200 μl. Samples were boiled and electrophoretically separated on a 10% SDS-polyacrylamide gel. Protein was transferred to PVDF membrane (Amersham) using a semi-dry transfer system (Bio-Rad). Blots were blocked in 5% skim milk in TPBS for one hour. Primary α-Rad17 antibody (Santa Cruz) was used at 1:500. Secondary goat α-rabbit IgG conjugated to horseradish peroxidase (Santa Cruz) was applied at 1:1000. Blots were developed using an enhanced chemiluminescence kit (Amersham).

In order to confirm mRNA translation suppression indicative of successful RNA interference, cellular levels of Rad17 protein were quantified. Cells from the pMACS-K/k-H1 and pMACS-K/k-H1-Rad17 positive populations were harvested at 48 and 72 hours post-transfection for Western Blotting. Our results indicate that the pMACS-K/k-H1-Rad17 plasmid expressing Rad17-specific shRNAs significantly reduced cellular Rad17 protein levels up to 72 hours after plasmid transfection (FIG. 3). Importantly, Rad17 levels were reduced relative to the pMACS-K/k-H1 transfected control, showing that Rad17 knock-down was attributable to shRNA expression (FIG. 3).

### EXAMPLE II

A plasmid expressing enhanced green fluorescence protein (EGFP) or a variant thereof is constructed wherein expression of EGFP is driven by a constitutive promoter recognized by the host cell. In addition, a tetracycline-inducible promoter system (tet system) is operably linked to an inverted repeat sequence (shRNA) with stem sequences complementary to a mRNA sequence derived from the gene of interest, for example, hoxb13.

For example, a sequence from the gene of interest (e.g., hoxb13; ccaggagctc cctgaaaccc (SEQ ID NO:3)), a loop sequence (e.g., a six to nine nucleotide loop sequence), the complement of the sequence from the gene of interest (e.g., gggtttcagg gagctcctgg (SEQ ID NO:4)), and transcriptional terminator are operably linked to the tet promoter. Thus, the tet promoter is operably linked to provide inducible expression of the small hairpin RNA of hoxb13. Thus, a shRNA is encoded by the tet system.

The tet promoter is well known in the art. The tetracycline-dependent regulatory system (tet system) is based upon the interaction between the tetracycline transactivator (tTA), consisting of the prokaryotic TetR fused to the activator domain of the herpes simplex virus VP16 protein, and the tetracycline-responsive element (TRE), consisting of seven copies of the prokaryotic tetracycline operator site (tetO) fused to a minimal CMV promoter. In the presence of tetracycline (tet), tTA loses its ability to bind the TRE and expression is shut off.

The tet system shRNA and EGFP expression system is transfected into primary neuronal cells. The cells are cultured in the presence of tet, allowing recovery and expression of the selection marker, but preventing expression of the shRNA, and the cells are then separated by flow cytometry. The sorted cells, expressing EGFP, are then assayed for effect of the siRNA, for example, by culture in the absence of tet, to determine if hoxb13 may act as an inhibitor of neuronal cell proliferation. The sorted cells may be split into two pools, those expressing EGFP and those lacking EGFP, and compared, wherein the cells lacking EGFP may serve as a control. In another embodiment, cells expressing EGFP but without the shRNA may be used as a control.

### EXAMPLE III

An inverted repeat operably linked to an RNA polymerase III (polIII) promoter of the U6 small nuclear RNA gene (U6) is constructed. The U6 driven inverted repeat and a selection marker (e.g., the ABC transporter gene MDR 1) are cloned into a nucleic acid sequence. The nucleic acid sequence is packaged in vitro using four recombinant SV40 proteins (VP1, VP2, VP3, and agno) or VP1 only (Kimchi-Sarfaty et al. (2003), High Cloning Capacity of In Vitro Packaged SV40 Vectors with No SV40 Virus Sequences, Hum. Gene Ther. 14(2):167-177). The in vitro packaged SV40 is infected into host cells.

Antibodies specific to the transporter are used to detect cell surface expression and cells expressing the transporter are sorted by fluorescence-activated cell sorter analysis (FACS). Sorted cells, expressing the selection marker are assayed for the effect of gene silencing by the inverted repeat sequence.

### EXAMPLE IV

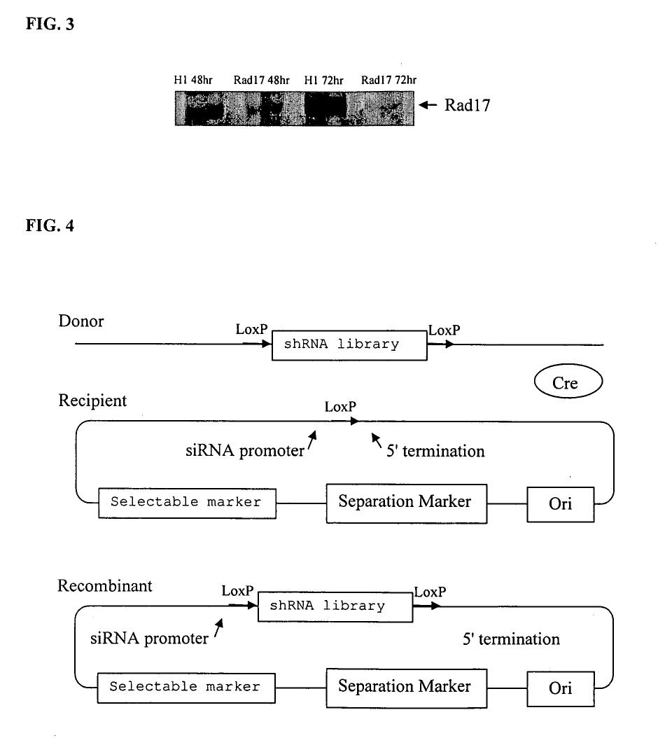

A library of siRNA producing plasmids are created using Lox/cre mediated recombination. For example, to overcome the cell type limitation imposed by a retroviral construct, a library of shRNA sequences, such as those of Paddison et al. (2004), are transferred to a plasmid of the invention. FIG. 4 illustrates a method of transferring a library of shRNA sequences, such as those of Paddison et al. (2004), to a plasmid of the present invention.

The library of shRNA sequences are transformed into 5×10⁸ cells and sorted using an autoMACS magnetic cell sorter (Miltenyi Biotech.) as described herein. The cells positive for expression of the separation marker are put back into culture and allowed to recover for 24 hours. The positive cells are then screened for a desired activity.

Alternatively, a separation marker may be introduced into the retroviral vector, for example, the retroviral vector of Paddison et al. (2004), allowing separation of infected from non-infected cells. Such a method may allow the use of a lower multiplicity of infection and/or reduce undesirable background.

All references, including publications, patents, and patent applications, cited herein are hereby incorporated by reference to the same extent as if each reference were individually and specifically indicated to be incorporated by reference and were set forth in its entirety herein.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A method of obtaining cells capable of expressing inhibitory RNA, the method comprising:
introducing at least one nucleic acid into a cell, wherein the at least one nucleic acid is able to express an RNAi molecule and a separation marker;
expressing a separation marker;
sorting the cell based on expression of the separation marker; and
expressing the RNAi molecule in an amount sufficient to inhibit expression of the target gene.

2. The method according to claim 1, wherein the RNAi molecule is a target gene selected from the group consisting of a cellular gene, an endogenous gene, a transgene and a viral gene.

3. The method according to claim 1, wherein the cell is of mammalian or plant origin.

4. The method according to claim 1, wherein sorting the cell comprises fluorescence activated cell sorting.

5. The method according to claim 1, wherein sorting the cell comprises magnetic cell separation.

6. The method according to claim 1, wherein introducing the at least one nucleic acid into the cell comprises introducing the at least one nucleic acid into less than 50% of the cells.

7. The method according to claim 1, wherein introducing at least one nucleic acid into a cell comprises introducing a library of RNAi molecules.

8. A method of enriching a population of mammalian cells having an inhibitory RNA sequence, the method comprising:
providing mammalian cells containing a target gene, wherein target cells are susceptible to RNA interference, and the target gene is expressed in the target cells;
introducing in the mammalian cells an RNA molecule, wherein the RNA molecule may produce RNA interference and contains a double-stranded area with a first region having a sequence which corresponds to a nucleotide sequence of the target gene and a second region having a sequence which is complementary to the first region, wherein the first and the second regions hybridize to each other to form a double-stranded RNA molecule;
introducing in the mammalian cells a separation marker;
expressing the separation marker in the mammalian cells, wherein expression of the separation marker is indicative of the presence of the double-stranded RNA molecule; and
sorting the mammalian cells expressing the separation marker, thereby enriching for the mammalian cells having the double-stranded RNA sequence.

9. The method according to claim 8, wherein the mammalian cells are of human origin.

10. The method according to claim 7 or claim 8, wherein the mammalian cells are primary cells.

11. he method according to claim 8, wherein the double-stranded ribonucleic acid structure is at least 18 bases in length and each of the ribonucleic acid strands is able to specifically hybridize to a deoxyribonucleic acid strand of the target gene over the at least 18 bases.

12. The method according to claim 10, further comprising inhibiting expression of the target gene by at least 10%.

13. The method according to claim 8, further comprising transfecting the mammalian cells with a first nucleotide sequence encoding the separation marker and a second nucleotide sequence capable of producing the RNA molecule.

14. The method according to claim 12, wherein transfecting the mammalian cells produces less than 40% of the mammalian cells expressing the separation marker.

15. A recombinant nucleic acid sequence comprising at least one nucleic acid sequence, wherein the at least one nucleic acid sequence is capable of expressing a separation marker and the at least one nucleic acid sequence comprises a promoter operably linked to a sequence capable of producing an inhibitor RNA, wherein the inhibitor RNA comprises a sequence complementary to a target gene.

16. The recombinant nucleic acid sequence of claim 15, wherein the inhibitor RNA comprises a first region which is complementary to the target gene and a second region which is complementary to the first region, wherein the inhibitor RNA is capable of forming a short hairpin RNA.

17. The recombinant nucleic acid sequence of claim 15, wherein the separation marker and the sequence capable of producing the inhibitor RNA are on the same nucleic acid molecule.

18. The recombinant nucleic acid sequence of claim 17, wherein the at least one nucleic acid is a vector.

19. The recombinant nucleic acid sequence of claim 18, wherein the vector comprises pHYPER.

20. The recombinant nucleic acid sequence of claim 15, wherein the separation marker is recognized by an antibody.

21. The recombinant nucleic acid sequence of claim 20, wherein the antibody is coupled to a label, the label selected from the group consisting of a magnetic reagent, a fluorescent molecule, an enzyme, and combinations of any thereof.

22. The recombinant nucleic acid sequence of claim 15, wherein the promoter is a type III DNA dependent RNA polymerase promoter.

23. The recombinant nucleic acid sequence of claim 15, wherein the inhibitor RNA comprises a library of sequences capable of forming a short hairpin RNA.

24. A cell comprising the recombinant nucleic acid sequence of claim 15.

25. A recombinant nucleic acid sequence comprising:
at least one nucleic acid sequence having means for producing a separation marker and means for producing a ribonucleic acid sequence which is able to inhibit translation of a target gene.


SEQUENCE LISTING (4 sequences)

SEQ ID NO: 1 - DNA, 64 bp, Artificial
  top strand of the Rad 17 shRNA sequence
gatcccccagactgggttgacccatcttcaagagagatgggtcaacccagtctgtttttggaaa

SEQ ID NO: 2 - DNA, 64 bp, Artificial
  Complementary strand shown as the bottom strand of figure 1
tcgaaaaggtttttgtctgacccaactgggtagagagaacttctacccagttgggtcagacccc

SEQ ID NO: 3 - DNA, 20 bp, Mus musculus
ccaggagctccctgaaaccc

SEQ ID NO: 4 - DNA, 20 bp, Artificial
  Complement of the hoxb13 sequence
gggtttcagggagctcctgg